(19) United States
(12) Patent Application Publication
(10) Pub. No.: US 2011/0112157 A1
(43) Pub. Date: May 12, 2011

(54) PROCESS FOR THE PREPARATION OF ZOLMITRIPTAN, SALTS AND SOLVATES THEREOF

(75) Inventors: Debashish Datta, Maharashtra (IN); Vinayak G. Gore, Maharashtra (IN); Maheshkumar S. Gadakar, Maharashtra (IN); Kiran Pokharkar, Maharashtra (IN); Kiran Phatangare, Maharashtra (IN)

(73) Assignee: GENERICS [UK] LIMITED, Hertfordshire (GB)

(21) Appl. No.: 12/681,000

(22) PCT Filed: Oct. 3, 2008
(86) PCT No.: PCT/GB2008/050906

§ 371 Date: Jan. 14, 2011

(30) Foreign Application Priority Data
Oct. 3, 2007 (IN) 1959/MUM/2007

(51) Int. Cl. A61K 31/422; C07D 413/02; A61P 25/06

(52) U.S. Cl. 514/376; 548/229

(57) ABSTRACT

The present invention relates to an improved process for the preparation of the active pharmaceutical ingredient zolmitriptan. In particular, it relates to an efficient process for the preparation of zolmitriptan and its pharmaceutically acceptable salts and solvates.

### CROSS-REFERENCE TO RELATED APPLICATION(s)

This application is a Section 371 National Stage Application of International No. PCT/GB2008/050906, filed 3 Oct. 2008 and published as WO 2009/044211 A1 on 9 Apr. 2009, which claims priority from the IN Patent Application No. 1959/MUM/2007, filed 3 Oct. 2007, the contents of which are incorporated herein in their entirety for all purposes.

### FIELD OF THE INVENTION

The present invention relates to an improved process for the preparation of the active pharmaceutical ingredient zolmitriptan. In particular, it relates to an efficient process for the preparation of zolmitriptan and its pharmaceutically acceptable salts and solvates.

### BACKGROUND OF THE INVENTION

Zolmitriptan (I), chemically named (4S)-4-[[3-[2-(dimethylamino)ethyl]-1H-indol-5-yl]methyl]-2-oxazolidinone, is a selective serotonin 5-hydroxytryptamine-1D (5-HT1D) receptor agonist and is currently marketed for the acute treatment of the headache phase of migraine attacks, with or without aura.

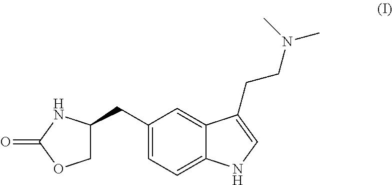

Zolmitriptan is structurally derived from tryptamine. Its therapeutic activity in treating migraine headache may be attributed to its agonist effects at 5-HT1B and 5-HT1D receptors on the extracerebral intracranial blood vessels that are thought to become dilated during an attack and on the trigeminal sensory nerves that innervate them. Activation of these 5-HT1B and 5-HT1D receptors may result in constriction of pain-producing intracranial blood vessels and inhibition of neuropeptide release that leads to decreased inflammation in sensitive tissues and reduced central trigeminal pain signal transmission.

Various processes for the preparation of zolmitriptan are disclosed in the prior art.

A process to obtain zolmitriptan base (I), and a pharmaceutically acceptable solvate thereof, is disclosed in U.S. Pat. No. 5,466,699 and is illustrated in Schemes 1, 1A and 1B. The process described is based on a Fischer indole synthesis, comprising the following steps. 4-Nitro-(L)-phenylalanine (II) was sequentially converted into (S)-4-(4-aminobenzyl)-1,3-oxazolidin-2-one hydrochloride (VI), which was further diazotized by treatment with sodium nitrite in conc. HCl to afford an intermediate diazonium salt, which was reduced to afford (S)-4-(4-hydrazinobenzyl)-1,3-oxazolidin-2-one hydrochloride (VII) using stannous chloride as a reducing agent (Scheme 1).

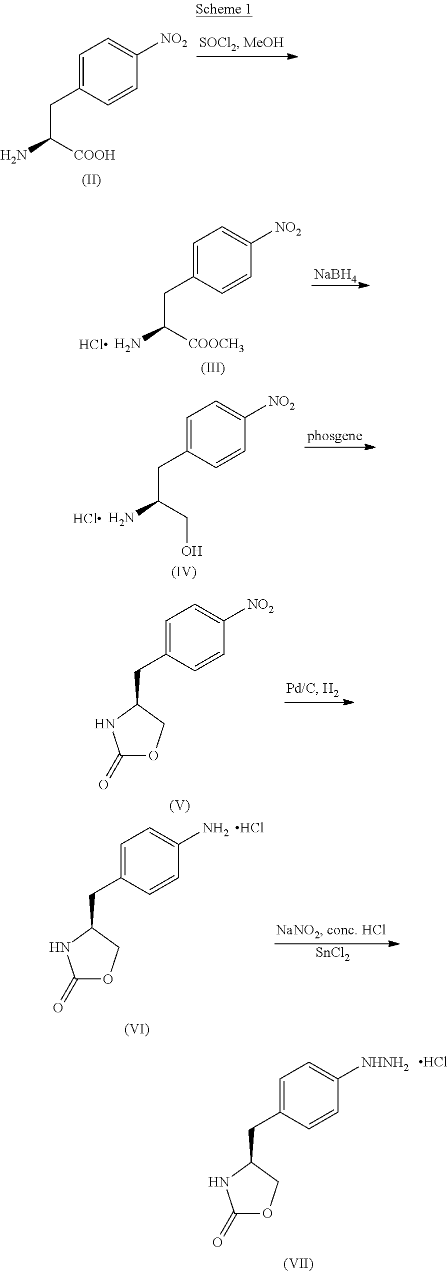

The isolated (S)-4-(4-hydrazinobenzyl)-1,3-oxazolidin-2-one hydrochloride (VII) was then condensed with 4-chloro-butyraldehyde dimethyl acetal and subjected to amination to afford (S)-2-[5-(2-oxa-1,3-oxazolidin-4-ylmethyl)-1H-indol-3-yl]ethylamine (VIII). After isolation by column chromatography purification, the amine (VIII) was converted into zolmitriptan (I) under Eschweiler-Clarke reaction conditions using formaldehyde, sodium cyanoborohydride and acetic acid. After the usual aqueous work-up procedures, zolmitriptan (I) was obtained as oil, which was further crystallized from isopropanol (Scheme 1A).

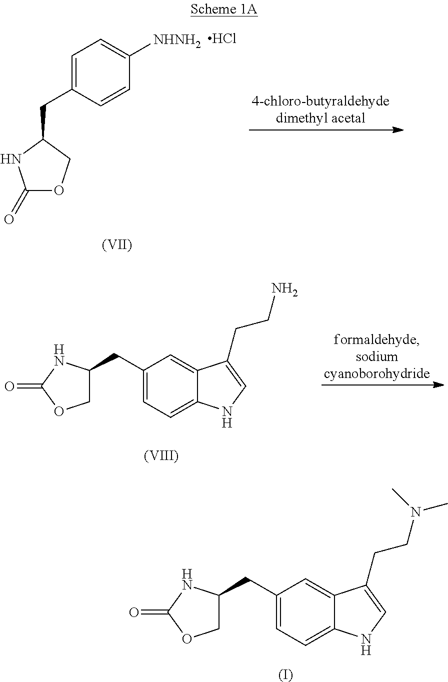

Alternatively, the isolated (S)-4-(4-hydrazinobenzyl)-1,3-oxazolidin-2-one hydrochloride (VII) was condensed with 4-N,N-dimethylamino-butyraldehyde diethyl acetal under Fischer indole reaction conditions in acetic acid and zolmitriptan (I) was isolated as an oil after column chromatography purification. The zolmitriptan oil obtained was further crystallized as an isopropanol solvate from isopropanol (Scheme 1B).

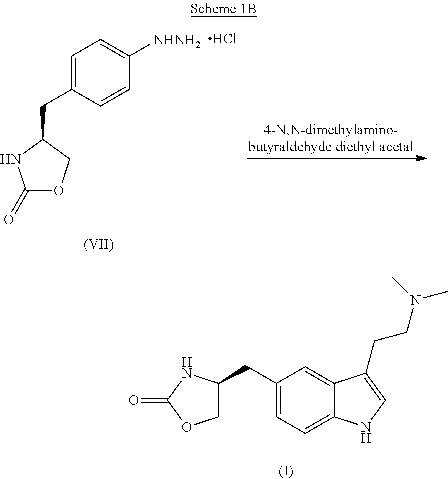

The major disadvantages of the process in U.S. Pat. No. 5,466,699 are as follows:
The process conditions reported for the preparation of the compounds (S)-4-(4-hydrazinobenzyl)-1,3-oxazolidin-2-one hydrochloride (VII), (S)-2-[5-(2-oxa-1,3-oxazolidin-4-ylmethyl)-1H-indol-3-yl]ethylamine (VIII) and zolmitriptan (I) are very low yielding (18% w/w) due to the formation of side products and degradation impurities.
The use of phosgene requires extensive safety precautions and is not a convenient reagent for use in commercial production.
Excess quantities of reagents, for example conc. HCl (˜18.5 vol.) and SnCl2 (8.0 eq.), are used which leads to degradation and the formation of impurities. Therefore the process needs chromatographic purification and results in very low yields.
The cyclisation reaction conditions are demanding, for example temperatures of 100-105° C. (acetic acid-water reflux) and very high volumes (acetic acid 52 vol., and water 156 vol.) are used. This also causes degradation of zolmitriptan.
The (S)-4-(4-hydrazinobenzyl)-1,3-oxazolidin-2-one hydrochloride (VII) intermediate was isolated by distillation of water.

An improvement to this process is disclosed in WO 97/06162, where conversion of 4-nitro-(L)-phenylalanine (II) to (S)-4-(4-aminobenzyl)-1,3-oxazolidin-2-one (IX) was carried out in one pot by avoiding the use of phosgene. The (S)-4-(4-aminobenzyl)-1,3-oxazolidin-2-one (IX) was further converted into zolmitriptan (I) in one pot by using sodium sulfite, which avoided the use of stannous chloride (Scheme 2).

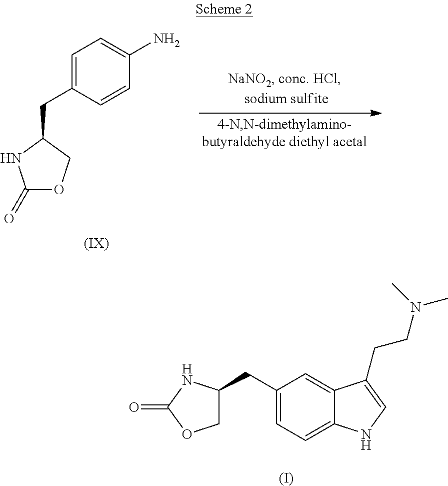

Although WO 97/06162 claims to disclose an improved one pot process to prepare zolmitriptan (I), it was observed that by following the process conditions described in WO 97/06162, the formation of degradation impurities was unavoidable, especially during the conversion of (S)-4-(4-aminobenzyl)-1,3-oxazolidin-2-one (IX) to the hydrazine intermediate and during the cyclisation to form zolmitriptan (I). (S)-4-(4-Hydrazinobenzyl)-1,3-oxazolidin-2-one was prepared at very acidic pH (˜1.0) which was responsible for the generation of unknown impurities. An additional disadvantage of the process lies in the high dilution of the reaction mass and extraction at elevated temperature. WO 97/06162 does not quote the yield of the zolmitriptan (I) obtained. The end product was obtained with yields of 30% and with high impurity content due to the one pot reaction. The process is therefore not applicable at industrial scale, either in terms of yield or purity.

The prior art publications WO 2004/014901 and US 2005/0245585 also disclose processes to prepare zolmitriptan (I).

The process illustrated in WO 2004/014901 is based on a Fischer indole synthesis from the hydrazone compound (XI), formed by the reaction of (S)-4-(4-hydrazinobenzyl)-1,3-oxazolidin-2-one (X) and α-keto-δ-valerolactone. The Fischer indole synthesis afforded the indole (XII). Further ring opening and transesterification of the indole (XII) gave the ester (XIII). The hydroxyl group of ester (XIII) was converted into a dimethylamino group and subsequent decarboxylation of the carboxylic acid moiety of compound (XIV) provided zolmitriptan (I) (Scheme 3).

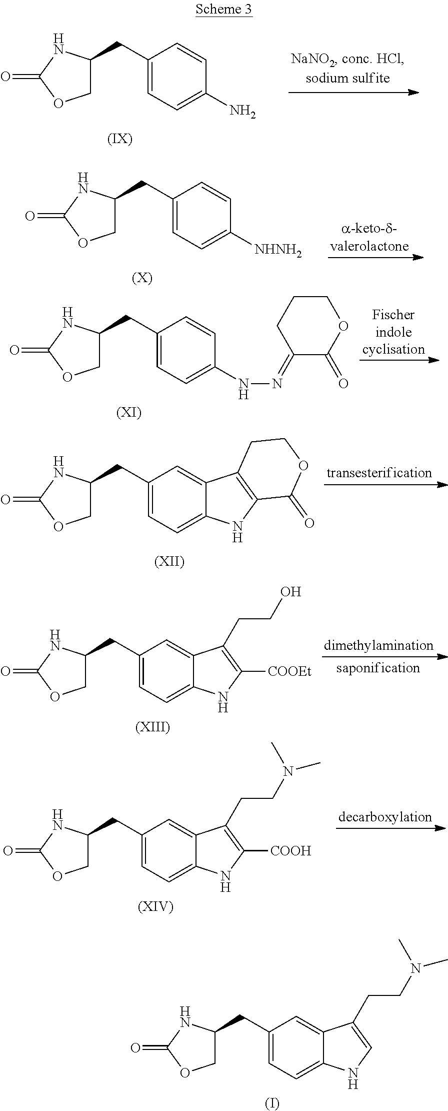

The major disadvantages of the process described in WO 2004/014901 are as follows:
It is a multi-step and lengthy process, which involves eight steps to obtain zolmitriptan (I) from (S)-4-(4-aminobenzyl)-1,3-oxazolidin-2-one (IX) which results in overall low yields.
The preparation of zolmitriptan (I) from the carboxylic acid (XIV) involves use of quinoline and cuprous oxide at very high temperature (200° C.), which can have an impact on the quality of the zolmitriptan (I) obtained and, in addition, such a high temperature would not be easily achieved on a commercial scale.
It is difficult to separate zolmitriptan (I) from quinoline, which is used as the solvent, due to their similar chemical characteristics.

Another process for the preparation of zolmitriptan is disclosed in WO 2005/105792. This publication discloses a process based on a Fischer indole synthesis and comprised the reaction of the diazo salt (XV) and alkyl-2-acetyl-5-(1,3-dioxo-2,3-dihydro-1H-2-isoindolyl)pentanoate (XVI) to afford a hydrazone intermediate (XVII). This was followed by the Fischer indole cyclisation of the hydrazone (XVII) to give indole product (XVIII), which after hydrolysis provided primary amine compound (XIX). The primary amine (XIX) was then methylated to obtain compound (XX), which was further decarboxylated to afford zolmitriptan (I) (Scheme 4).

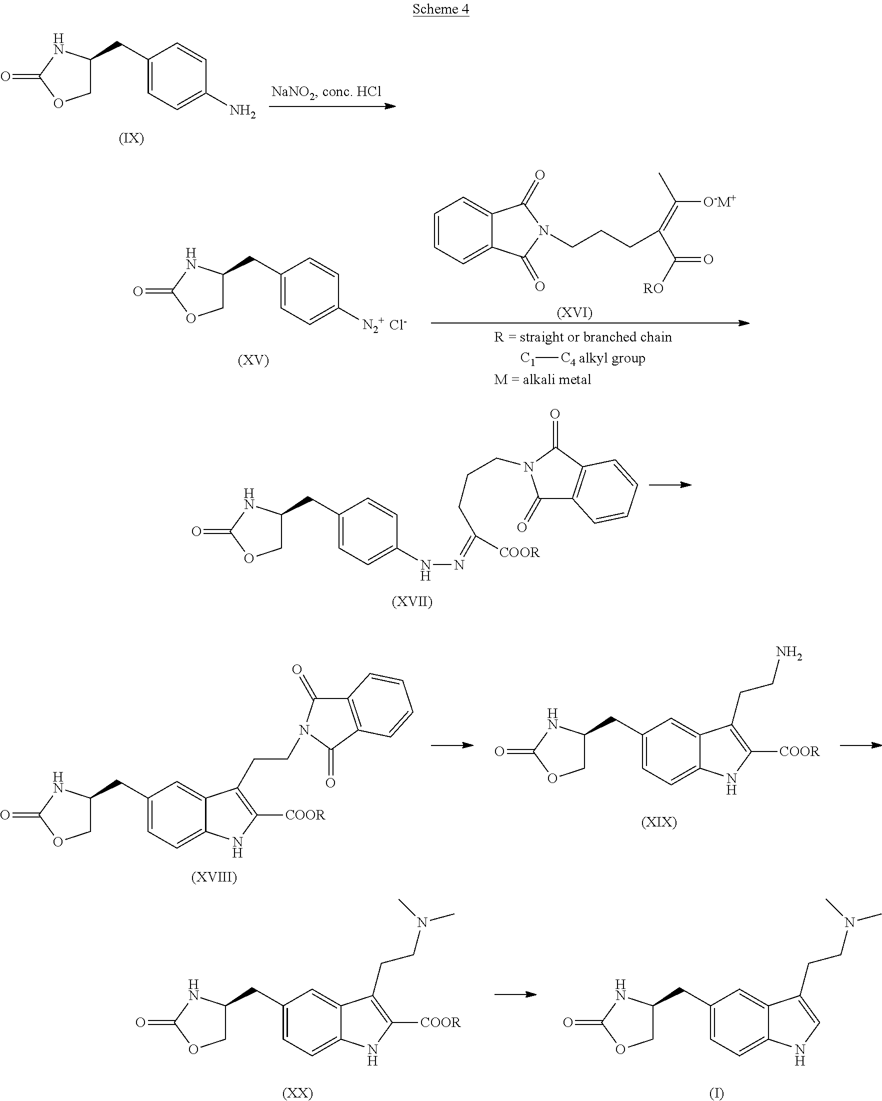

The major disadvantages of the process described in WO 2005/105792 are as follows:
It is a multi-step and lengthy process involving 5-7 steps to obtain zolmitriptan (I) from the diazo salt (XV) of (S)-4-(4-aminobenzyl)-1,3-oxazolidin-2-one (IX).
The work-up procedures involve highly basic conditions, which may cleave the oxazolidinone ring.
The overall yield reported is only 9-22%.

Consequently, the processes disclosed in the prior art for the preparation of zolmitriptan (I) suffer from various disadvantages, such as multi-step process, low yield, low purity, and difficulties to scale up to commercial scale. In view of the importance acquired by zolmitriptan (I) for the treatment of migraine, there is therefore a great need for developing a simple, inexpensive and commercially feasible process for the synthesis of zolmitriptan (I) with commercially acceptable yield and high purity.

### SUMMARY OF THE INVENTION

According to a first aspect of the present invention, there is provided a process for the preparation of zolmitriptan (I), comprising the steps of:

(a) diazotization of (S)-4-(4-aminobenzyl)-1,3-oxazolidin-2-one (IX), or a protected form thereof, to form a diazonium intermediate (XV), followed by reduction of the diazonium intermediate to give (S)-4-(4-hydrazinobenzyl)-1,3-oxazolidin-2-one (X), or a protected form thereof;
(b) condensation of (S)-4-(4-hydrazinobenzyl)-1,3-oxazolidin-2-one (X), or a protected form thereof, with 4-N,N-dimethylamino-butyraldehyde, or a protected form thereof, to form a hydrazone intermediate, or a protected form thereof; and
(c) cyclisation of the resultant hydrazone intermediate to yield zolmitriptan (I).

The diazotization in step (a) is preferably carried out using sodium nitrite, preferably using in excess of 1 equivalent of sodium nitrite. Preferably the sodium nitrite is allowed to react with the (S)-4-(4-aminobenzyl)-1,3-oxazolidin-2-one (IX), or the protected form thereof, for at least 1 hour, preferably at least 2 hours, preferably at least 3 hours, preferably for up to 4 hours, prior to the reduction of the diazonium intermediate (XV).

Preferably in step (a) the reduction of the diazonium intermediate (XV) is carried out using stannous chloride. Preferably the reduction is carried out under acidic conditions, preferably at a pH of about 1-3, preferably at a pH of about 2. Preferably the reduction is carried out using less than 5 equivalents of stannous chloride, preferably less than 4 equivalents, preferably less than 3 equivalents, preferably less than 2 equivalents, preferably using at least 1 equivalent of stannous chloride. Preferably the reduction is carried out at a temperature in the range of −10 to 65° C., preferably in the range of −10 to 5° C.

Preferably, after completion of the reduction of the diazonium intermediate (XV), the pH of the reaction mixture is adjusted to about pH 8-14, more preferably to about pH 8-9.

(S)-4-(4-Hydrazinobenzyl)-1,3-oxazolidin-2-one (X) is then condensed with 4-N,N-dimethylamino-butyraldehyde, or a protected form thereof, to form a hydrazone intermediate.

Preferably the (S)-4-(4-hydrazinobenzyl)-1,3-oxazolidin-2-one (X), or the protected form thereof, is not isolated prior to the condensation with 4-N,N-dimethylamino-butyraldehyde, or the protected form thereof.

Preferably the condensation of the (S)-4-(4-hydrazinobenzyl)-1,3-oxazolidin-2-one (X), or the protected form thereof, is carried out using at least 1.5 equivalents, preferably at least 2 equivalents, preferably at least 3 equivalents of 4-N,N-dimethylamino-butyraldehyde, or the protected form thereof.

Preferably the 4-N,N-dimethylamino-butyraldehyde is used in the form of an acetal, such as a dialkyl acetal. Most preferably, the acetal is the dimethyl acetal.

Preferably the 4-N,N-dimethylamino-butyraldehyde, or the protected form thereof, is combined with the (S)-4-(4-hydrazinobenzyl)-1,3-oxazolidin-2-one (X), or the protected form thereof, at a pH of greater than 5, preferably at a pH of greater than 7, preferably at a pH of greater than 8, preferably at a pH of greater than 9.

Preferably the condensation of the (S)-4-(4-hydrazinobenzyl)-1,3-oxazolidin-2-one (X), or the protected form thereof, with 4-N,N-dimethylamino-butyraldehyde, or the protected form thereof, to form a hydrazone intermediate is carried out at about pH 0-3. More preferably the condensation is carried out at approximately pH 2.

Preferably the condensation of the (S)-4-(4-hydrazinobenzyl)-1,3-oxazolidin-2-one (X), or the protected form thereof, with 4-N,N-dimethylamino-butyraldehyde, or the protected form thereof, is carried out at a temperature of −10 to 100° C., preferably 25-30° C.

Preferably the cyclisation of the hydrazone intermediate is carried out at acidic pH, more preferably at about pH 0-6, more preferably at about pH 0-3, and more preferably at approximately pH 2.

Preferably the cyclisation of the hydrazone intermediate is carried out at a temperature of −10 to 110° C., more preferably at 85-95° C.

Preferably the condensation and cyclisation reactions are carried out at relatively high dilution. Typically the dilution is 10-100 volumes, preferably 20-60 volumes, preferably 30-50 volumes, but more preferably the dilution is approximately 50 volumes.

Preferably the cyclisation is carried out in the presence of one or more mineral acids or Lewis acids selected from hydrochloric acid, sulfuric acid, acetic acid, phosphoric acid, boron trifluoride, and trifluoroacetic anhydride.

Preferably the zolmitriptan (I) obtained in step (c) is isolated by the following steps:

(a) washing the reaction mixture at acidic pH with one or more organic solvents or mixtures thereof;
(b) basification of the reaction mixture, removal of solid by-products, and extraction of zolmitriptan (I) by using one or more organic solvents or mixtures thereof;
(c) washing the zolmitriptan (I) organic solvent extract with water; and optionally
(d) purification of the zolmitriptan (I) organic solvent extract using a solid adsorbent.

Preferably the one or more organic solvents or mixtures thereof used in isolation step (a) or (b) are selected from acetates such as ethyl acetate, methyl acetate, isopropyl acetate; chlorinated hydrocarbon solvents such as dichloromethane, chloroform, dichloroethane; ethers such as diethyl ether, tertiary butyl methyl ether, diisopropyl ether; or aliphatic hydrocarbons such as hexane, heptane, pentane; or mixtures thereof.

Preferably in isolation step (b) the reaction mixture is basified to about pH 8-14, more preferably to approximately pH 8-9. Preferably the reaction mixture is basified using a metal carbonate, such as sodium carbonate or potassium carbonate.

Preferably the isolation process comprises step (d). Preferably the solid adsorbent used in isolation step (d) is activated carbon.

Optionally, the process of the first aspect of the invention can include a further step for the preparation of zolmitriptan (I) by using one or more organic solvents selected from acetates such as ethyl acetate, methyl acetate, isopropyl acetate; chlorinated hydrocarbon solvents such as dichloromethane, chloroform, dichloroethane; ethers such as diethyl ether, tertiary butyl methyl ether, diisopropyl ether; ketonic solvents such as acetone, methyl ethyl ketone, diethyl ketone, methyl isopropyl ketone and other higher ketones (such as methyl n-propyl ketone, 2-methylheptan-3-one, 6-undecanone, 5-methyl-5-hexen-2-one); alcoholic solvents such as methanol, ethanol, n-propanol, t-butanol, pentanols or higher alcohols (such as n-amyl alcohol, n-hexanol, 2-phenylethanol); or mixtures thereof.

Optionally, the process of the first aspect of the invention can include a further step for the purification of zolmitriptan (I) by crystallizing from one or more organic solvents selected from acetates such as ethyl acetate, methyl acetate, isopropyl acetate; chlorinated hydrocarbon solvents such as dichloromethane, chloroform, dichloroethane; ethers such as diethyl ether, tertiary butyl methyl ether, diisopropyl ether; ketonic solvents such as acetone, methyl ethyl ketone, diethyl ketone, methyl isopropyl ketone and other higher ketones (such as methyl n-propyl ketone, 2-methylheptan-3-one, 6-undecanone, 5-methyl-5-hexen-2-one); alcoholic solvents such as methanol, ethanol, n-propanol, t-butanol, pentanols or higher alcohols (such as n-amyl alcohol, n-hexanol, 2-phenylethanol); or mixtures thereof.

Preferably the isolation and purification of zolmitriptan (I), prepared by a process according to the first aspect of the invention, is done without using chromatographic purification. Preferably the isolation and purification of zolmitriptan (I) comprises the use of organic or inorganic acids capable of forming acid addition salts. Typically, the organic or inorganic acids used are benzoic, oxalic, succinic, hydrochloric, hydrobromic, acetic, propionic, maleic, formic or a sulfonic acid. Preferably, the acid is succinic acid.

Optionally, another aspect of the invention can include a further step for the preparation of a pharmaceutically acceptable solvate of zolmitriptan (I). Preferably the solvate prepared is the isopropyl acetate, tertiary butyl acetate, chloroform, dichloromethane, diethyl ketone, methyl isopropyl ketone, diisopropyl ether, diethyl ether, n-pentanol, allyl alcohol, benzyl alcohol, phenyl butanol, cyclopentanol, cyclohexanol, n-pentane, heptane, cyclopentane or cyclohexane solvate.

Optionally, another aspect of the invention can include a further step for the preparation of a pharmaceutically acceptable salt of zolmitriptan (I). Preferably the salt prepared is a pharmaceutically acceptable acid addition salt with benzoic, oxalic, succinic, hydrochloric, hydrobromic, acetic, propionic, maleic, fumaric, formic, sulfonic, phosphoric, malic, citric, sulfuric, lactic or tartaric acid.

Preferably in the first aspect of the invention, the process is a ‘one pot’ process, i.e. a process in which none of the intermediates in the preparation of zolmitriptan (I) are isolated and/or purified.

Preferably the process of the first aspect of the invention provides zolmitriptan (I) with an HPLC purity of greater than 99%, 99.5%, 99.8%, or 99.9%.

Preferably the process of the first aspect of the invention provides zolmitriptan (I) from (S)-4-(4-aminobenzyl)-1,3-oxazolidin-2-one (IX) in a yield of 35%, 40%, 45% or more.

Preferably the process of the first aspect of the invention provides zolmitriptan (I) on an industrial scale, preferably in batches of 100 g, 500 g, 1 kg, 5 kg, 10 kg, 50 kg, 100 kg, 200 kg, 500 kg, or more.

A second aspect of the invention provides zolmitriptan (I) prepared by a process according to the first aspect of the invention.

A third aspect of the invention provides zolmitriptan (I) of greater than 99% purity (as measured by HPLC). Preferably the zolmitriptan of the current invention has a purity of greater than 99.5%, more preferably greater than 99.8%, and most preferably greater than 99.9%. Pure zolmitriptan was crystallized from organic solvents such as alcoholic or ketonic solvents, preferably isopropanol, diethyl ketone, methyl isopropyl ketone, to achieve a high quality zolmitriptan (I) (HPLC purity of more than 99.9%).

Preferably the zolmitriptan (I) of the second or third aspect of the invention is suitable for treating or preventing migraine, headache, cluster headache, or headache associated with vascular disorders.

A fourth aspect of the invention provides a solvate of zolmitriptan (I) selected from the isopropyl acetate, tertiary butyl acetate, chloroform, dichloromethane, diethyl ketone, methyl isopropyl ketone, diisopropyl ether, diethyl ether, n-pentanol, allyl alcohol, benzyl alcohol, phenyl butanol, cyclopentanol, cyclohexanol, n-pentane, heptane, cyclopentane or cyclohexane solvate. Preferably the solvate of zolmitriptan (I) is suitable for treating or preventing migraine, headache, cluster headache, or headache associated with vascular disorders.

A fifth aspect of the invention provides a pharmaceutical composition comprising zolmitriptan (I) prepared by a process according to the first aspect of the invention, or comprising zolmitriptan (I) according to the second, third or fourth aspect of the invention. Preferably the pharmaceutical composition is suitable for treating or preventing migraine, headache, cluster headache, or headache associated with vascular disorders.

A sixth aspect of the invention provides a method of treating or preventing migraine, headache, cluster headache, or headache associated with vascular disorders, comprising administering to a patient in need thereof a therapeutically or prophylactically effective amount of zolmitriptan (I) prepared by a process according to the first aspect of the invention or zolmitriptan (I) according to the second, third or fourth aspect of the invention.

A seventh aspect of the invention provides the use of zolmitriptan (I) prepared by a process according to the first aspect of the invention or zolmitriptan (I) according to the second, third or fourth aspect of the invention, in the preparation of a medicament for the treatment of migraine, headache, cluster headache, or headache associated with vascular disorders.

The present invention thus provides an improved process for the preparation of highly pure zolmitriptan (I). The improved process is simple, inexpensive, good yielding and can be easily adopted for commercial production with a high degree of consistency and reproducibility.

The present process offers a very good yield of zolmitriptan (I) (45% w/w) even though stannous chloride is used for the reduction of the diazonium salt (XV) to hydrazine (X). The improvement in yield was achieved by controlling the pH, temperature, dilution and reaction time in the present ‘one pot’ process.

The present process offers a very high purity of zolmitriptan (I) (more than 99.5%) without chromatographic purification.

The present invention provides a process for the synthesis of zolmitriptan (I) which minimizes degradation.

The present invention also provides a process for the synthesis of zolmitriptan (I) with a very high yield and/or high purity.

The present invention further provides a process for the synthesis of zolmitriptan (I) which is adaptable for large scale commercial production.

The present invention provides a high quality zolmitriptan (I) and pharmaceutically acceptable solvates and salts thereof.

The present invention additionally provides a pharmaceutical composition comprising zolmitriptan (I) obtained by the improved process.

### DETAILED DESCRIPTION OF THE INVENTION

The term ‘zolmitriptan’ as used herein throughout the description and claims means zolmitriptan and/or any salt, solvate, hydrate or enantiomer thereof or a mixture of any of these.

The present invention provides an improved, convenient process for the synthesis of zolmitriptan, preferably in ‘one pot’, and preferably using stannous chloride for the reduction of the diazonium intermediate (XV). The term ‘one pot’ process as used herein throughout the description and claims does not mean that the whole reaction is necessarily carried out in one and the same pot, instead the term ‘one pot’ process means that none of the intermediates in the preparation of zolmitriptan (I) are isolated and/or purified. For example, the reaction mixture in one pot may be added to a reagent in another pot, i.e. using two pots in total but only one pot at a time, without isolating and/or purifying any intermediates.

The present inventors have observed that the advantages of the present invention are:
1. The time for the formation of hydrazine (X) is reduced from 18 hours in the prior art to 7-8 hours.
2. The present process achieves a much higher overall yield: 45% compared to 9-30% in the prior art.
3. The present process achieves an excellent quality: a purity of zolmitriptan (I) of more than 99.9%, which easily meets the ICH guidelines.
4. No chromatographic purification is required, therefore the present process is easily scalable.

The present inventors have further observed that there is a strict control on the formation of impurities during the entire process. The total impurities were controlled to less than 0.50% in the crude zolmitriptan (I). The crude zolmitriptan (I) obtained had an HPLC purity of between 99.1-99.7%, typically of more than 99.5%, consistently.

A preferred embodiment of the improved ‘one pot’ synthesis according to the invention is outlined in Scheme 5.

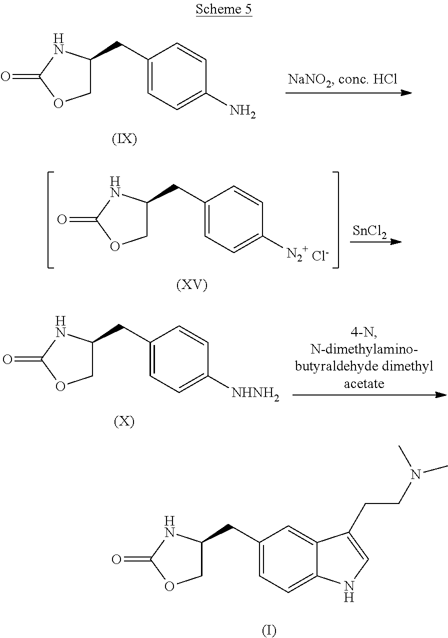

Diazotization of (S)-4-(4-aminobenzyl)-1,3-oxazolidin-2-one (IX) was carried out using sodium nitrite (˜2 equivalents) in the presence of hydrochloric acid at low temperatures (5 to −10° C.) to give the diazonium intermediate (XV). The diazotization is preferably carried out using sodium nitrite, preferably using in excess of 1 equivalent of sodium nitrite. It is necessary to continue the reaction at lower temperature (5 to −10° C.) for 3-4 hours to achieve a complete conversion of (S)-4-(4-aminobenzyl)-1,3-oxazolidin-2-one (IX) into the diazonium intermediate (XV). It was observed that if the reaction was terminated before 3-4 hours, unreacted (S)-4-(4-aminobenzyl)-1,3-oxazolidin-2-one (IX) was found as a major impurity in the final isolated crude zolmitriptan (I).

The diazonium intermediate (XV) was then reduced using stannous chloride to achieve an efficient and fast conversion to (S)-4-(4-hydrazinobenzyl)-1,3-oxazolidin-2-one (X).

To obtain an efficient reduction of the diazonium intermediate (XV) to (S)-4-(4-hydrazinobenzyl)-1,3-oxazolidin-2-one (X), the quantity of stannous chloride, the mode of addition of stannous chloride, the reaction temperature, and the reaction time were optimized.

The inventors have surprisingly found that a solution of stannous chloride in hydrochloric acid at lower temperature (5 to −10° C.) afforded a clean conversion of the diazonium salt (XV) to hydrazine (X).

Typically, stannous chloride (˜2 equivalents) was dissolved in concentrated hydrochloric acid (˜4 volumes) and was cooled to 5 to −10° C., preferably −5 to −10° C. To this solution, the diazonium intermediate was added at 5 to −10° C. (preferably −5 to −10° C.) and maintained for 4 hours. The diazonium intermediate (XV) was cleanly converted into hydrazine (X) and under these conditions the formation of degradation impurities was controlled within the desired limits.

The reduction is preferably carried out using less than 3 equivalents of stannous chloride, and more preferably using 2 or less equivalents of stannous chloride.

The reduction of the diazonium intermediate is preferably carried out at a temperature of −10 to 65° C., more preferably at −10 to 5° C.

The pH of the (S)-4-(4-hydrazinobenzyl)-1,3-oxazolidin-2-one (X) solution was adjusted to approximately pH 8-9 from around pH 2 by using metal carbonates, preferably by using sodium carbonate, at 25-30° C. After pH adjustment, the reaction mixture was further diluted with water in such a way that the total volume of the reaction mixture was in the range of 40 to 150 volumes, preferably about 40 to 50 volumes, preferably about 50 volumes. To this solution, 4-N,N-dimethylamino-butyraldehyde dimethyl acetal (˜3 equivalents) was added at 25-30° C. and the pH of the reaction mixture was adjusted to pH 2 with dilute HCl at 25-30° C. After pH adjustment, the reaction mixture was stirred at 25-30° C. for 1 hour to complete the hydrazone formation. The hydrazone was preferably not isolated.

For the purposes of the present invention, ‘volumes dilution’ means the quantity of solvent used relative to the starting material. For example, if 100 g of (S)-4-(4-aminobenzyl)-1,3-oxazolidin-2-one (IX) are used as starting material and the reaction is carried out at 20 volumes dilution, this means that 100×20=2000 ml solvent are used.

The pH adjustment of the hydrazine solution from pH 2 to approximately pH 8-9, the dilution of the hydrazine solution (40-100 volumes), and the further pH adjustment after the addition of 4-N,N-dimethylamino-butyraldehyde dimethyl acetal are believed to minimize the formation of degradation impurities. Also, the preparation of the hydrazone at lower temperatures (25-30° C.) is believed to control degradation before the conversion into crude zolmitriptan (I).

After completion of the hydrazone formation the reaction mixture was further heated to 85-90° C. for 4-5 hours to achieve a complete conversion of the hydrazone intermediate to zolmitriptan (I).

Preferably the process of the first aspect of the invention can include an additional step, wherein the zolmitriptan (I) is isolated by modified work-up procedures to eliminate or minimize the degradation impurities or chemical impurities formed, which comprises the following steps:

(a) washing of the reaction mixture at acidic pH with one or more organic solvents or mixtures thereof;
(b) basification of the reaction mixture, removal of solid by-products (e.g. stannous salts), and extraction of zolmitriptan (I) by using one or more organic solvents or mixtures thereof;
(c) washing of the zolmitriptan (I) organic solvent extract with water; and optionally
(d) purification of the zolmitriptan (I) organic solvent extract using a solid adsorbent.

The reaction mixture was cooled to 25-30° C. and washed with organic solvents, preferably with ethyl acetate (10 volumes). These washings at acidic pH (approximately pH 2) eliminate unreacted intermediates and degradation impurities. The reaction mixture pH was then readjusted to approximately pH 8-9 using a metal carbonate, preferably sodium carbonate.

The solid by-products, typically stannous salts, formed during the reaction and after pH adjustment were separated preferably by filtration (Celite®). This assisted in the isolation of zolmitriptan (I) in relatively pure form.

The crude zolmitriptan (I) was extracted into organic solvents, preferably ethyl acetate, and washed several times with water to remove residual stannous salts and 4-N,N-dimethylamino-butyraldehyde.

Distillation of the ethyl acetate afforded zolmitriptan ethyl acetate solvate with an HPLC purity of more than 99%. Highly pure zolmitriptan (I) was obtained from this solvate by crystallization using isopropanol (HPLC purity of more than 99.55%).

Alternatively zolmitriptan was easily isolated as a highly pure (HPLC purity of 99.0%) solvate or solvate-free in good yield using different solvents or mixtures of solvents e.g. methanol, ethanol, n-propanol, t-butanol, pentanols, acetone, methyl ethyl ketone, diethyl ketone, methyl isopropyl ketone and other higher ketones (such as methyl n-propyl ketone, 2-methylheptan-3-one, 6-undecanone, 5-methyl-5-hexen-2-one), diethyl ether, tertiary butyl methyl ether, diisopropyl ether, hexane, heptane and pentane. The ketonic solvents form solvates efficiently and selectively with zolmitriptan, which not only affords a higher yield (45-50% w/w) but also a higher purity (HPLC purity of 99.0%).

Alternatively, zolmitriptan (I) was also purified by converting it into a suitable acid addition salt such as the benzoate, succinate, maleate etc. The preferred way to purify the zolmitriptan base is via its succinate salt. A typical procedure for this purification is described below.

To the ethyl acetate extracts, after washing with water, an aqueous solution of succinic acid was added. The zolmitriptan succinate formed remained in the aqueous layer leaving the impurities in the ethyl acetate layer. The aqueous solution of zolmitriptan succinate was separated and further washed with ethyl acetate to assure the complete removal of impurities. The pH of the aqueous solution of zolmitriptan succinate was adjusted to approximately pH 8-9 from about pH 2-3 by using a metal carbonate, preferably sodium carbonate. The zolmitriptan free base thus formed was extracted into ethyl acetate as a solvate and crystallized from isopropanol to achieve the required quality (HPLC purity of more than 99.90%).

The process according to the first aspect of the invention can be used for the preparation of zolmitriptan (I) or a pharmaceutically acceptable salt or solvate of zolmitriptan (I).

Further zolmitriptan solvates were also prepared using organic solvents such as alcoholic or ketonic solvents, preferably isopropanol, diethyl ketone, methyl isopropyl ketone etc., which were converted to pure zolmitriptan (I) without chromatographic purification.

Further pure zolmitriptan (I) was crystallized from organic solvents such as alcoholic or ketonic solvents, preferably isopropanol, diethyl ketone, methyl isopropyl ketone, to achieve a high quality zolmitriptan (I) (HPLC purity of more than 99.9%).

Following a process of the first aspect of the invention, zolmitriptan (I) (HPLC purity of more than 99%) was isolated as a free-flowing crystalline off-white solvate.

High quality zolmitriptan (I) and pharmaceutically acceptable solvates thereof are used for the manufacture of a medicament for the treatment of migraine, headache, cluster headache, or headache associated with vascular disorders.

The following paragraphs enumerated consecutively from 1 through 65 provide for various aspects of the present invention. In one embodiment, the present invention provides:

1. A process for the preparation of zolmitriptan (I), comprising:
(a) diazotization of (S)-4-(4-aminobenzyl)-1,3-oxazolidin-2-one (IX), or a protected form thereof, to form a diazonium intermediate (XV), followed by reduction of the diazonium intermediate to give (S)-4-(4-hydrazinobenzyl)-1,3-oxazolidin-2-one (X), or a protected form thereof;
(b) condensation of (S)-4-(4-hydrazinobenzyl)-1,3-oxazolidin-2-one (X), or a protected form thereof, with 4-N,N-dimethylamino-butyraldehyde, or a protected form thereof, to form a hydrazone intermediate, or a protected form thereof; and
(c) cyclisation of the resultant hydrazone intermediate to yield zolmitriptan (I).
2. The process as claimed in paragraph 1, wherein the diazotization of (S)-4-(4-aminobenzyl)-1,3-oxazolidin-2-one (IX), or the protected form thereof, is carried out using sodium nitrite.
3. The process as claimed in paragraph 2, wherein the diazotization is carried out using in excess of 1 equivalent of sodium nitrite.
4. The process as claimed in paragraph 1 or 2, wherein the sodium nitrite is allowed to react with the (S)-4-(4-aminobenzyl)-1,3-oxazolidin-2-one (IX), or the protected form thereof; for at least 1 hour prior to the reduction of the diazonium intermediate (XV).
5. The process as claimed in any one of the preceding paragraphs, wherein the reduction of the diazonium intermediate (XV) is carried out using stannous chloride.
6. The process as claimed in paragraph 5, wherein the reduction is carried out under acidic conditions.
7. The process as claimed in paragraph 5 or 6, wherein the reduction is carried out using less than 5 equivalents of stannous chloride.
8. The process as claimed in paragraph 7, wherein the reduction is carried out using 2 or less equivalents of stannous chloride.
9. The process as claimed in any one of paragraphs 5 to 8, wherein the reduction is carried out using at least 1 equivalent of stannous chloride.
10. The process as claimed in any one of the preceding paragraphs, wherein the reduction of diazonium intermediate (XV) is carried out at a temperature in the range of −10 to 65° C.
11. The process as claimed in paragraph 10, wherein the reduction of diazonium intermediate (XV) is carried out at a temperature in the range of −10 to 5° C.
12. The process as claimed in any one of the preceding paragraphs, wherein after completion of the reduction of the diazonium intermediate (XV), the pH of the reaction mixture is adjusted to pH 8-14.
13. The process as claimed in paragraph 12, wherein the pH of the reaction mixture is adjusted to pH 8-9.
14. The process as claimed in any one of the preceding paragraphs, wherein the (S)-4-(4-hydrazinobenzyl)-1,3-oxazolidin-2-one (X), or the protected form thereof, is not isolated prior to the condensation with 4-N,N-dimethylamino-butyraldehyde, or the protected form thereof
15. The process as claimed in any one of the preceding paragraphs, wherein the condensation of the (S)-4-(4-hydrazinobenzyl)-1,3-oxazolidin-2-one (X), or the protected form thereof, is carried out using at least 1.5 equivalents of 4-N,N-dimethylamino-butyraldehyde, or the protected form thereof.
16. The process as claimed in any one of the preceding paragraphs, wherein the 4-N,N-dimethylamino-butyraldehyde is used in the form of an acetal.
17. The process as claimed in paragraph 16, wherein the acetal is a dialkyl acetal.
18. The process as claimed in paragraph 17, wherein the dialkyl acetal is the dimethyl acetal.
19. The process as claimed in any one of the preceding paragraphs, wherein the 4-N,N-dimethylamino-butyraldehyde, or the protected form thereof, is combined with the (S)-4-(4-hydrazinobenzyl)-1,3-oxazolidin-2-one (X), or the protected form thereof, at a pH of greater than 5.
20. The process as claimed in any one of the preceding paragraphs, wherein the condensation of the (S)-4-(4-hydrazinobenzyl)-1,3-oxazolidin-2-one (X), or the protected form thereof, with 4-N,N-dimethylamino-butyraldehyde, or the protected form thereof, to form a hydrazone intermediate is carried out at pH 0-3.
21. The process as claimed in paragraph 20, wherein the condensation of the (S)-4-(4-hydrazinobenzyl)-1,3-oxazolidin-2-one (X), or the protected form thereof, is carried out at approximately pH 2.
22. The process as claimed in any one of the preceding paragraphs, wherein the condensation of the (S)-4-(4-hydrazinobenzyl)-1,3-oxazolidin-2-one (X), or the protected form thereof, with 4-N,N-dimethylamino-butyraldehyde, or the protected form thereof, to form a hydrazone intermediate is carried out at a temperature of −10 to 100° C.
23. The process as claimed in any one of the preceding paragraphs, wherein the cyclisation of the hydrazone intermediate is carried out at acidic pH.
24. The process as claimed in paragraph 23, wherein the cyclisation of the hydrazone intermediate is carried out at pH 0-3.
25. The process as claimed in paragraph 24, wherein the cyclisation of the hydrazone intermediate is carried out at approximately pH 2.
26. The process as claimed in any one of the preceding paragraphs, wherein the cyclisation of the hydrazone intermediate is carried out at a temperature of −10 to 110° C.
27. The process as claimed in paragraph 26, wherein the cyclisation of the hydrazone intermediate is carried out at 85-95° C.
28. The process as claimed in any one of the preceding paragraphs, wherein the condensation of the (S)-4-(4-hydrazinobenzyl)-1,3-oxazolidin-2-one (X), or the protected form thereof, with 4-N,N-dimethylamino-butyraldehyde, or the protected form thereof, to form a hydrazone intermediate, and the cyclisation of the hydrazone intermediate are carried out at relatively high dilution.
29. The process as claimed in paragraph 28, wherein the dilution is 10-100 volumes.
30. The process as claimed in paragraph 29, wherein the dilution is approximately 50 volumes.
31. The process as claimed in any one of the preceding paragraphs, wherein the cyclisation of the hydrazone intermediate is carried out in the presence of one or more mineral acids or Lewis acids.
32. The process as claimed in paragraph 31, wherein the one or more mineral acids or Lewis acids are selected from hydrochloric acid, sulfuric acid, acetic acid, phosphoric acid, boron trifluoride, and trifluoroacetic anhydride.
33. The process as claimed in any one of the preceding paragraphs, wherein the zolmitriptan (I) obtained in step (c) is isolated by the following steps:
(a) washing the reaction mixture at acidic pH with one or more organic solvents or mixtures thereof;
(b) basification of the reaction mixture, removal of solid by-products, and extraction of zolmitriptan (I) by using one or more organic solvents or mixtures thereof;
(c) washing the zolmitriptan (I) organic solvent extract with water; and optionally
(d) purification of the zolmitriptan (I) organic solvent extract using a solid adsorbent.
34. The process as claimed in paragraph 33, wherein the one or more organic solvents or mixtures thereof used in isolation step (a) or (b) are selected from acetates such as ethyl acetate, methyl acetate, isopropyl acetate; chlorinated hydrocarbon solvents such as dichloromethane, chloroform, dichloroethane; ethers such as diethyl ether, tertiary butyl methyl ether, diisopropyl ether; or aliphatic hydrocarbons such as hexane, heptane, pentane; or mixtures thereof.
35. The process as claimed in paragraph 33 or 34, wherein in isolation step (b) the reaction mixture is basified to pH 8-14.
36. The process as claimed in paragraph 35, wherein the reaction mixture is basified to approximately pH 8-9.
37. The process as claimed in any one of paragraphs 33 to 36, wherein in isolation step (b) the reaction mixture is basified using a metal carbonate.
38. The process as claimed in paragraph 37, wherein the metal carbonate is sodium carbonate or potassium carbonate.
39. The process as claimed in any one of paragraphs 33 to 38, wherein the solid adsorbent used in isolation step (d) is activated carbon.
40. The process as claimed in any one of the preceding paragraphs, further comprising a step for the preparation of zolmitriptan (I) by using one or more organic solvents selected from acetates such as ethyl acetate, methyl acetate, isopropyl acetate; chlorinated hydrocarbon solvents such as dichloromethane, chloroform, dichloroethane; ethers such as diethyl ether, tertiary butyl methyl ether, diisopropyl ether; ketonic solvents such as acetone, methyl ethyl ketone, diethyl ketone, methyl isopropyl ketone and other higher ketones; alcoholic solvents such as methanol, ethanol, n-propanol, t-butanol, pentanols or higher alcohols; or mixtures thereof.
41. The process as claimed in any one of the preceding paragraphs, further comprising a step for the purification of zolmitriptan (I) by crystallizing from one or more organic solvents selected from acetates such as ethyl acetate, methyl acetate, isopropyl acetate; chlorinated hydrocarbon solvents such as dichloromethane, chloroform, dichloroethane; ethers such as diethyl ether, tertiary butyl methyl ether, diisopropyl ether; ketonic solvents such as acetone, methyl ethyl ketone, diethyl ketone, methyl isopropyl ketone and other higher ketones; alcoholic solvents such as methanol, ethanol, n-propanol, t-butanol, pentanols or higher alcohols; or mixtures thereof.
42. The process as claimed in any one of the preceding paragraphs, further comprising a step for the purification of zolmitriptan (I) comprising the use of an organic or inorganic acid capable of forming an acid addition salt.
43. The process as claimed in paragraph 42, wherein the organic or inorganic acid used is benzoic, oxalic, succinic, hydrochloric, hydrobromic, acetic, propionic, maleic, formic or a sulfonic acid.
44. The process as claimed in paragraph 43, wherein the organic or inorganic acid used is succinic acid.
45. The process as claimed in any one of the preceding paragraphs, further comprising a step for the preparation of a pharmaceutically acceptable solvate of zolmitriptan (I).
46. The process as claimed in paragraph 45, wherein the solvate prepared is the isopropyl acetate, tertiary butyl acetate, chloroform, dichloromethane, diethyl ketone, methyl isopropyl ketone, diisopropyl ether, diethyl ether, n-pentanol, allyl alcohol, benzyl alcohol, phenyl butanol, cyclopentanol, cyclohexanol, n-pentane, heptane, cyclopentane or cyclohexane solvate.
47. The process as claimed in any one of the preceding paragraphs, further comprising a step for the preparation of a pharmaceutically acceptable salt of zolmitriptan (I).
48. The process as claimed in paragraph 47, wherein the pharmaceutically acceptable salt is the benzoic, oxalic, succinic, hydrochloric, hydrobromic, acetic, propionic, maleic, fumaric, formic, sulfonic, phosphoric, malic, citric, sulfuric, lactic or tartaric acid salt.
49. The process as claimed in any one of the preceding paragraphs, wherein the process is a ‘one pot’ process.
50. The process as claimed in any one of the preceding paragraphs, wherein none of the intermediates in the preparation of zolmitriptan (I) are isolated and/or purified.
51. The process as claimed in any one of the preceding paragraphs, wherein the process is carried out without chromatographic purification.
52. The process as claimed in any one of the preceding paragraphs, wherein the process provides zolmitriptan (I) with an HPLC purity of greater than 99%.
53. The process as claimed in any one of the preceding paragraphs, wherein the process provides zolmitriptan (I) from (S)-4-(4-aminobenzyl)-1,3-oxazolidin-2-one (IX) in a yield of 35% or more.
54. The process as claimed in any one of the preceding paragraphs, wherein the process provides zolmitriptan (I) on an industrial scale in batches of 100 g or more.
55. Zolmitriptan (I) prepared by a process as claimed in any one of the preceding paragraphs.
56. Zolmitriptan (I) with an HPLC purity of greater than 99%.
57. Zolmitriptan (I) as claimed in paragraph 56, with an HPLC purity of greater than 99.5%.
58. Zolmitriptan (I) as claimed in paragraph 57, with an HPLC purity of greater than 99.8%.
59. Zolmitriptan (I) as claimed in paragraph 58, with an HPLC purity of greater than 99.9%.
60. The zolmitriptan (I) as claimed in any one of paragraphs 55 to 59 for treating or preventing migraine, headache, cluster headache, or headache associated with vascular disorders.
61. A solvate of zolmitriptan (I) selected from the isopropyl acetate, tertiary butyl acetate, chloroform, dichloromethane, diethyl ketone, methyl isopropyl ketone, diisopropyl ether, diethyl ether, n-pentanol, allyl alcohol, benzyl alcohol, phenyl butanol, cyclopentanol, cyclohexanol, n-pentane, heptane, cyclopentane or cyclohexane solvate.
62. The solvate of zolmitriptan (I) as claimed in paragraph 61 for treating or preventing migraine, headache, cluster headache, or headache associated with vascular disorders.
63. A pharmaceutical composition comprising zolmitriptan (I) as claimed in any one of paragraphs 55 to 62.
64. The pharmaceutical composition as claimed in paragraph 63 for treating or preventing migraine, headache, cluster headache, or headache associated with vascular disorders.
65. A method of treating or preventing migraine, headache, cluster headache, or headache associated with vascular disorders, comprising administering a therapeutically or prophylactically effective amount of zolmitriptan (I) as claimed in any one of paragraphs 55 to 62 to a patient in need thereof.

Further details of the invention are illustrated below in the following non-limiting examples.

### EXAMPLES

### Example 1

### Preparation of Zolmitriptan (I)

(S)-4-(4-Aminobenzyl)-1,3-oxazolidin-2-one (IX) (100 g) was charged in water (400 ml, 4.0 volumes) and conc. HCl (200 ml, 2.0 volumes) was added at 25-30° C. The solution was cooled to 5 to −10° C. and a solution of sodium nitrite (54 g, 1.5 equivalents) in water (400 ml, 4.0 volumes) was added whilst maintaining the temperature below −5° C. After completion of the addition, the reaction mixture was stirred for 3 hours resulting in the formation of the diazonium chloride (XV) in solution.

The solution of diazonium chloride (XV) was slowly added to a pre-dissolved solution of stannous chloride (234.5 g, 2.0 equivalents in 200 ml, 2.0 volumes of HCl) at 5 to −10° C. The mixture was stirred for 4 hours at 5 to −10° C. After completion of the hydrazine formation (as measured by TLC), the pH of the reaction mixture was adjusted to pH 8-9 from about pH 2 by using sodium carbonate (100 g) at 25-30° C. to afford a solution of (S)-4-(4-hydrazinobenzyl)-1,3-oxazolidin-2-one (X).

The solution of (S)-4-(4-hydrazinobenzyl)-1,3-oxazolidin-2-one (X) was further diluted with water (2.0 L, 20 volumes). After dilution, 4-N,N-dimethylamino-butyraldehyde dimethyl acetal (246.0 g, 2.5 equivalents) was added to the hydrazine solution at 25-30° C. and the pH of the reaction mixture was checked (pH=9). The pH of the reaction mixture was adjusted to pH 2 by slow addition of 50% (v/v) HCl solution (˜50 ml, 0.5 volume). The reaction mixture was stirred for 1 hour at pH 2 to achieve a complete conversion to the corresponding hydrazone (as confirmed by TLC).

The reaction mixture was then heated at 85-90° C. for a further 4-5 hours to achieve a complete conversion of the hydrazone to zolmitriptan (I).

The reaction mixture was cooled to 25-30° C. and washed with ethyl acetate (2×μL, 2×10 volumes) at pH 2. The pH of the aqueous layer obtained was adjusted with sodium carbonate to about pH 8-9. The metal salts formed during the reaction and after pH adjustment were separated by filtration through a Celite® bed which was further washed with ethyl acetate (2×1 L, 2×10 volumes). The aqueous filtrate obtained was extracted with ethyl acetate (1 L, 10 volumes) at about pH 8-9 and the combined ethyl acetate extracts and washings obtained at about pH 8-9 were further washed with water (3×2 L, 3×20 volumes). Then the ethyl acetate layer was treated with activated carbon (Norit Supra activated charcoal, 10 g, 10% w/w) for 1 hour at 25-30° C. The charcoal was separated by filtration through a Celite® bed and zolmitriptan (I) was easily isolated as the ethyl acetate solvate by distillation of the ethyl acetate on a rotary evaporator at 45-50° C. at 50-100 mbar.

The obtained zolmitriptan ethyl acetate solvate was further dissolved in isopropanol (500 ml) and 200 ml of the isopropanol was distilled off at 45-50° C. at 50-100 mbar. The isopropanol solution of zolmitriptan was further stirred at 0 to −10° C. for 3 hours before the product was filtered and washed with chilled isopropanol (200 ml, 2.0 volumes). The product was dried at 55-60° C. for 6 hours to achieve a constant weight.

Yield: 45% (w/w)

m.p.: 137-141° C.

HPLC purity: 99.10%

By following similar experimental conditions, zolmitriptan (I) was also isolated, optionally as a solvate, by using different solvents or mixtures of solvents, e.g. methanol, ethanol, n-propanol, t-butanol, pentanols, acetone, methyl ethyl ketone, diethyl ketone, methyl isopropyl ketone and other higher ketones (such as methyl n-propyl ketone, 2-methylheptan-3-one, 6-undecanone, 5-methyl-5-hexen-2-one), diethyl ether, tertiary butyl methyl ether, diisopropyl ether, hexane, heptane, pentane etc. The yields obtained were in the range of 35-45% with an HPLC purity in the range of 99.10-99.7%.

### Example 1

### Crystallization of Zolmitriptan (I)

The pure zolmitriptan (I) obtained above was crystallized from isopropanol as follows. Pure zolmitriptan (40.0 g) was dissolved in isopropanol (200 ml) at 45-50° C. to obtain a clear solution. To the clear solution, Norit Supra B activated carbon (4.0 g, 10% w/w) was added and the mixture heated for 1 hour at 45-50° C. Then the solution was filtered through a Celite® bed and the filtrate was concentrated under reduced pressure to ˜100 ml. The resulting suspension was cooled to 0-5° C. and stirred for 1 hour. The crystallized zolmitriptan (I) was filtered and dried at 45-50° C. under reduced pressure until a constant weight was obtained (around 6 hours).

Yield: 87% (w/w)

HPLC purity: 99.94%

By following similar experimental conditions, pure zolmitriptan (I) was crystallized by using different solvents or mixtures of solvents, e.g. methanol, ethanol, n-propanol, t-butanol, pentanols, acetone, methyl ethyl ketone, diethyl ketone, methyl isopropyl ketone and other higher ketones (such as methyl n-propyl ketone, 2-methylheptan-3-one, 6-undecanone, 5-methyl-5-hexen-2-one), diethyl ether, tertiary butyl methyl ether, diisopropyl ether, hexane, heptane and pentane. The yields obtained were in the range of 85-95% with an HPLC purity in the range of 99.7 to 99.95%.

1-86. (canceled)

87. A process for the preparation of zolmitriptan (I), comprising:
(a) diazotization of (S)-4-(4-aminobenzyl)-1,3-oxazolidin-2-one (IX), or a protected form thereof, to form a diazonium intermediate (XV), followed by reduction of the diazonium intermediate to give (S)-4-(4-hydrazinobenzyl)-1,3-oxazolidin-2-one (X), or a protected form thereof;
(b) condensation of (S)-4-(4-hydrazinobenzyl)-1,3-oxazolidin-2-one (X), or a protected form thereof, with 4-N,N-dimethylamino-butyraldehyde, or a protected form thereof, to form a hydrazone intermediate, or a protected form thereof; and
(c) cyclisation of the resultant hydrazone intermediate to yield zolmitriptan (I).

88. The process as claimed in claim 87, wherein:
(i) the diazotization of (S)-4-(4-aminobenzyl)-1,3-oxazolidin-2-one (IX), or the protected form thereof, is carried out using sodium nitrite; and/or
(ii) the diazotization of (S)-4-(4-aminobenzyl)-1,3-oxazolidin-2-one (IX), or the protected form thereof, is carried out using in excess of 1 equivalent of sodium nitrite; and/or
(iii) the diazotization of (S)-4-(4-aminobenzyl)-1,3-oxazolidin-2-one (IX), or the protected form thereof, is carried out using sodium nitrite, wherein the sodium nitrite is allowed to react with the (S)-4-(4-aminobenzyl)-1,3-oxazolidin-2-one (IX), or the protected form thereof, for at least 1 hour prior to the reduction of the diazonium intermediate (XV).

89. The process as claimed in claim 87, wherein:
(i) the reduction of the diazonium intermediate (XV) is carried out using stannous chloride; and/or
(ii) the reduction of the diazonium intermediate (XV) is carried out using stannous chloride under acidic conditions; and/or
(iii) the reduction of the diazonium intermediate (XV) is carried out using less than 5 equivalents of stannous chloride; and/or
(iv) the reduction of the diazonium intermediate (XV) is carried out using 2 or less equivalents of stannous chloride; and/or
(v) the reduction of the diazonium intermediate (XV) is carried out using at least 1 equivalent of stannous chloride; and/or
(vi) the reduction of the diazonium intermediate (XV) is carried out at a temperature in the range of −10 to 65° C.; and/or
(vii) the reduction of the diazonium intermediate (XV) is carried out at a temperature in the range of −10 to 5° C.; and/or
(viii) after completion of the reduction of the diazonium intermediate (XV), the pH of the reaction mixture is adjusted to pH 8-14; and/or
(ix) after completion of the reduction of the diazonium intermediate (XV), the pH of the reaction mixture is adjusted to pH 8-9.

90. The process as claimed in claim 87, wherein:
(i) the (S)-4-(4-hydrazinobenzyl)-1,3-oxazolidin-2-one (X), or the protected form thereof, is not isolated prior to the condensation with 4-N,N-dimethylamino-butyraldehyde, or the protected form thereof; and/or
(ii) the condensation of the (S)-4-(4-hydrazinobenzyl)-1,3-oxazolidin-2-one (X), or the protected form thereof, is carried out using at least 1.5 equivalents of 4-N,N-dimethylamino-butyraldehyde, or the protected form thereof; and/or
(iii) the condensation of the (S)-4-(4-hydrazinobenzyl)-1,3-oxazolidin-2-one (X), or the protected form thereof, is carried out using 4-N,N-dimethylamino-butyraldehyde in the form of an acetal; and/or
(iv) the condensation of the (S)-4-(4-hydrazinobenzyl)-1,3-oxazolidin-2-one (X), or the protected form thereof, is carried out using 4-N,N-dimethylamino-butyraldehyde in the form of a dialkyl acetal; and/or
(v) the condensation of the (S)-4-(4-hydrazinobenzyl)-1,3-oxazolidin-2-one (X), or the protected form thereof, is carried out using 4-N,N-dimethylamino-butyraldehyde in the form of the dimethyl acetal; and/or
(vi) the 4-N,N-dimethylamino-butyraldehyde, or the protected form thereof, is combined with the (S)-4-(4-hydrazinobenzyl)-1,3-oxazolidin-2-one (X), or the protected form thereof, at a pH of greater than 5; and/or
(vii) the condensation of the (S)-4-(4-hydrazinobenzyl)-1,3-oxazolidin-2-one (X), or the protected form thereof, with 4-N,N-dimethylamino-butyraldehyde, or the protected form thereof, to form the hydrazone intermediate is carried out at pH 0-3; and/or
(viii) the condensation of the (S)-4-(4-hydrazinobenzyl)-1,3-oxazolidin-2-one (X), or the protected form thereof, with 4-N,N-dimethylamino-butyraldehyde, or the protected form thereof, to form the hydrazone intermediate is carried out at approximately pH 2; and/or
(ix) the condensation of the (S)-4-(4-hydrazinobenzyl)-1,3-oxazolidin-2-one (X), or the protected form thereof, with 4-N,N-dimethylamino-butyraldehyde, or the protected form thereof, to form the hydrazone intermediate is carried out at a temperature of −10 to 100° C.

91. The process as claimed in claim 87, wherein:
(i) the cyclisation of the hydrazone intermediate is carried out at acidic pH; and/or
(ii) the cyclisation of the hydrazone intermediate is carried out at pH 0-3; and/or
(iii) the cyclisation of the hydrazone intermediate is carried out at approximately pH 2; and/or
(iv) the cyclisation of the hydrazone intermediate is carried out at a temperature of −10 to 110° C.; and/or
(v) the cyclisation of the hydrazone intermediate is carried out at a temperature of 85-95° C.; and/or
(vi) the condensation of the (S)-4-(4-hydrazinobenzyl)-1,3-oxazolidin-2-one (X), or the protected form thereof, with 4-N,N-dimethylamino-butyraldehyde, or the protected form thereof, to form the hydrazone intermediate, and the cyclisation of the hydrazone intermediate are carried out at relatively high dilution; and/or
(vii) the condensation of the (S)-4-(4-hydrazinobenzyl)-1,3-oxazolidin-2-one (X), or the protected form thereof, with 4-N,N-dimethylamino-butyraldehyde, or the protected form thereof, to form the hydrazone intermediate, and the cyclisation of the hydrazone intermediate are carried out at a dilution of 10-100 volumes; and/or
(viii) the condensation of the (S)-4-(4-hydrazinobenzyl)-1,3-oxazolidin-2-one (X), or the protected form thereof, with 4-N,N-dimethylamino-butyraldehyde, or the protected form thereof, to form the hydrazone intermediate, and the cyclisation of the hydrazone intermediate are carried out at a dilution of approximately 50 volumes; and/or
(ix) the cyclisation of the hydrazone intermediate is carried out in the presence of one or more mineral acids or Lewis acids; and/or
(x) the cyclisation of the hydrazone intermediate is carried out in the presence of one or more mineral acids or Lewis acids selected from hydrochloric acid, sulfuric acid, acetic acid, phosphoric acid, boron trifluoride, and trifluoroacetic anhydride.

92. The process as claimed in claim 87, wherein the zolmitriptan (I) obtained in step (c) is isolated by the following steps:
(a) washing the reaction mixture at acidic pH with one or more organic solvents or mixtures thereof;
(b) basification of the reaction mixture, removal of solid by-products, and extraction of zolmitriptan (I) by using one or more organic solvents or mixtures thereof;
(c) washing the zolmitriptan (I) organic solvent extract with water; and optionally
(d) purification of the zolmitriptan (I) organic solvent extract using a solid adsorbent.

93. The process as claimed in claim 92, wherein:
(i) the one or more organic solvents or mixtures thereof used in isolation step (a) or (b) are selected from acetates such as ethyl acetate, methyl acetate, isopropyl acetate; chlorinated hydrocarbon solvents such as dichloromethane, chloroform, dichloroethane; ethers such as diethyl ether, tertiary butyl methyl ether, diisopropyl ether; or aliphatic hydrocarbons such as hexane, heptane, pentane; or mixtures thereof; and/or
(ii) in isolation step (b) the reaction mixture is basified to pH 8-14; and/or
(iii) in isolation step (b) the reaction mixture is basified to approximately pH 8-9; and/or
(iv) in isolation step (b) the reaction mixture is basified using a metal carbonate; and/or
(v) in isolation step (b) the reaction mixture is basified using sodium carbonate or potassium carbonate; and/or
(vi) the solid adsorbent used in isolation step (d) is activated carbon.

94. The process as claimed in claim 87, further comprising a step for the preparation of zolmitriptan (I) by using one or more organic solvents selected from acetates such as ethyl acetate, methyl acetate, isopropyl acetate; chlorinated hydrocarbon solvents such as dichloromethane, chloroform, dichloroethane; ethers such as diethyl ether, tertiary butyl methyl ether, diisopropyl ether; ketonic solvents such as acetone, methyl ethyl ketone, diethyl ketone, methyl isopropyl ketone and other higher ketones; alcoholic solvents such as methanol, ethanol, n-propanol, t-butanol, pentanols or higher alcohols; or mixtures thereof.

95. The process as claimed in claim 87, further comprising a step for the purification of zolmitriptan (I) by crystallizing from one or more organic solvents selected from acetates such as ethyl acetate, methyl acetate, isopropyl acetate; chlorinated hydrocarbon solvents such as dichloromethane, chloroform, dichloroethane; ethers such as diethyl ether, tertiary butyl methyl ether, diisopropyl ether; ketonic solvents such as acetone, methyl ethyl ketone, diethyl ketone, methyl isopropyl ketone and other higher ketones; alcoholic solvents such as methanol, ethanol, n-propanol, t-butanol, pentanols or higher alcohols; or mixtures thereof.

96. The process as claimed in claim 87, further comprising a step for the purification of zolmitriptan (I) comprising:
(i) the use of an organic or inorganic acid capable of forming an acid addition salt; and/or
(ii) the use of an organic or inorganic acid capable of forming an acid addition salt, wherein the organic or inorganic acid used is benzoic, oxalic, succinic, hydrochloric, hydrobromic, acetic, propionic, maleic, formic or a sulfonic acid; and/or
(iii) the use of an organic or inorganic acid capable of forming an acid addition salt, wherein the organic or inorganic acid used is succinic acid.

97. The process as claimed in claim 87, further comprising a step for the preparation of:
(i) a pharmaceutically acceptable solvate of zolmitriptan (I); and/or
(ii) a pharmaceutically acceptable solvate of zolmitriptan (I), wherein the solvate prepared is the isopropyl acetate, tertiary butyl acetate, chloroform, dichloromethane, diethyl ketone, methyl isopropyl ketone, diisopropyl ether, diethyl ether, n-pentanol, allyl alcohol, benzyl alcohol, phenyl butanol, cyclopentanol, cyclohexanol, n-pentane, heptane, cyclopentane or cyclohexane solvate; and/or
(iii) a pharmaceutically acceptable salt of zolmitriptan (I); and/or
(iv) a pharmaceutically acceptable salt of zolmitriptan (I), wherein the pharmaceutically acceptable salt is the benzoic, oxalic, succinic, hydrochloric, hydrobromic, acetic, propionic, maleic, fumaric, formic, sulfonic, phosphoric, malic, citric, sulfuric, lactic or tartaric acid salt.

98. The process as claimed in claim 87, wherein:
(i) the process is a ‘one pot’ process; and/or
(ii) none of the intermediates in the preparation of zolmitriptan (I) are isolated and/or purified; and/or
(iii) the process is carried out without chromatographic purification; and/or
(iv) the process provides zolmitriptan (I) with an HPLC purity of greater than 99%; and/or
(v) the process provides zolmitriptan (I) from (S)-4-(4-aminobenzyl)-1,3-oxazolidin-2-one (IX) in a yield of 35% or more; and/or
(vi) the process provides zolmitriptan (I) on an industrial scale in batches of 100 g or more.

99. Zolmitriptan (I) prepared by a process as claimed in claim 87.

100. Zolmitriptan (I) with an HPLC purity of:
(i) greater than 99%; and/or
(ii) greater than 99.5%; and/or
(iii) greater than 99.8%; and/or
(iv) greater than 99.9%.

101. A solvate of zolmitriptan (I) selected from the isopropyl acetate, tertiary butyl acetate, chloroform, dichloromethane, diethyl ketone, methyl isopropyl ketone, diisopropyl ether, diethyl ether, n-pentanol, allyl alcohol, benzyl alcohol, phenyl butanol, cyclopentanol, cyclohexanol, n-pentane, heptane, cyclopentane or cyclohexane solvate.

102. A pharmaceutical composition comprising zolmitriptan (I) as claimed in claim 99.

103. A pharmaceutical composition comprising zolmitriptan (I) as claimed in claim 100.

104. A pharmaceutical composition comprising a solvate of zolmitriptan (I) as claimed in claim 101.

105. A method of treating or preventing migraine, headache, cluster headache, or headache associated with vascular disorders, comprising administering a therapeutically or prophylactically effective amount of zolmitriptan (I) as claimed in claim 99 to a patient in need thereof.

106. A method of treating or preventing migraine, headache, cluster headache, or headache associated with vascular disorders, comprising administering a therapeutically or prophylactically effective amount of zolmitriptan (I) as claimed in claim 100 to a patient in need thereof.

107. A method of treating or preventing migraine, headache, cluster headache, or headache associated with vascular disorders, comprising administering a therapeutically or prophylactically effective amount of a solvate of zolmitriptan (I) as claimed in claim 101 to a patient in need thereof.
